(19) United States
(12) Patent Application Publication
(10) Pub. No.: US 2013/0239961 A1
(43) Pub. Date: Sep. 19, 2013

(54) APPARATUS, COMPUTER PROGRAM, METHOD AND SYSTEM FOR PORTABLE BREATHING ASSISTANCE

(71) Applicant: SMARTMED USA INC., Kansas City, KS (US)

(72) Inventors: Charles Andrew Ross, JR., Kansas City, KS (US); Steve Phillips, Olathe, KS (US); James W. Farnham, IV, Olathe, KS (US); Warren B. Shy, Blanchard, OK (US)

(73) Assignee: SmartMed USA Inc., Kansas City, KS (US)

(21) Appl. No.: 13/832,581

(22) Filed: Mar. 15, 2013

Related U.S. Application Data
(60) Provisional application No. 61/611,660, filed on Mar. 16, 2012.
(60) Provisional application No. 61/733,768, filed on Dec. 5, 2012.

(51) Int. Cl. A61M 16/00; A61M 16/16; A61M 16/10

(52) U.S. Cl. CPC A61M 16/0057; A61M 16/105; A61M 16/16; A61M 16/0051
USPC 128/202.22; 128/204.23; 128/203.16

(57) ABSTRACT

An apparatus, a computer program, a method and a system for portable breathing assistance. The apparatus, computer program, method, and system are directed to a portable breathing system for providing pulmonary medical care to a patient, for obtaining a plurality of metrics associated with the breathing system and the patient, and for wirelessly communicating the metrics to one or more external computing devices.

### RELATED APPLICATION

This non-provisional patent application claims priority benefit, with regard to all common subject matter, of earlier filed U.S. Provisional Patent Application No. 61/733,768, filed Dec. 5, 2012, and entitled “APPARATUS, COMPUTER PROGRAM, AND SYSTEM FOR A PORTABLE BREATHING MACHINE,” and U.S. Provisional Patent Application No. 61/611,660, filed Mar. 16, 2012, and entitled “COMPUTER PROGRAM, METHOD, AND SYSTEM FOR ACQUIRING BATTERY METRICS AND FOR CALCULATING A BATTERY LIFE.” The identified earlier-filed provisional patent applications are hereby incorporated by reference in their entirety into the present non-provisional application.

### FIELD

Embodiments of the present invention are directed to an apparatus, computer program, method, and system for portable breathing assistance. In particular, the apparatus, computer program, method, and system are directed to a portable breathing system for providing pulmonary medical care to a patient, for obtaining a plurality of information relating to the breathing system and the patient, for wirelessly communicating the acquired information to one or more external computing devices, and for monitoring and analyzing the metrics via an external computing device.

### BACKGROUND

There are a few medical procedures available for treating persons who are unable to maintain clear airways, constant breathing, or oxygenation of blood. One such procedure is tracheal intubation, which requires an endotracheal tube to be passed into the mouth, through the vocal apparatus, and into the trachea. Because of the invasiveness of the procedure, tracheal intubation is often times associated with complications, such as laceration of the upper airway tissues, broken teeth, or other complications. In some instances, more serious complications can arise, such as pulmonary aspiration of stomach contents or intubation of the tube through the esophagus, which can each potentially lead to fatal anoxia.

There are very few non-invasive procedures available that can keep a person's airways open during medical emergencies. The procedures that are available may include the use of devices such as ventilators, continuous air-pressure application machines, or the like. However, these devices are commonly large in size and weight, and are thus generally restricted for use within large-scale medical facilities. In addition, such devices generally do not include smart, interactive features that allow users to communicate in real-time with other medical personnel and to distribute information regarding the devices and/or the patients.

### SUMMARY

Embodiments of the present invention include a portable breathing system for providing positive airway pressure to a patient experiencing a pulmonary medical issue, with the system comprising: an enclosure; a power source for providing electrical power to the breathing system; a fluid pump for maintaining positive airway pressure in an airway of the patient; a controller for controlling the fluid pump; a memory element for storing breathing system metrics; and a communications interface configured for wirelessly transmitting the breathing system metrics.

Embodiments of the present invention additionally include a method for monitoring breathing system metrics obtained from a portable breathing system, with the method comprising the following steps: providing the portable breathing system, with the portable breathing system including a fluid pump, a controller for controlling the fluid pump, and a communications interface for wirelessly transmitting the breathing system metrics; receiving the breathing system metrics form the portable breathing system; monitoring the breathing system metrics to determine whether a metric falls below a predefined threshold; and upon the metric falling below the predefined threshold, sending a notification to a user.

Embodiments of the present invention further include an additional portable breathing system for providing positive airway pressure to a patient experiencing a pulmonary medical issue, with the system comprising: an enclosure; a power source for providing electrical power to the breathing system; an oxygen source; a fluid pump for providing oxygen from the oxygen source and ambient air to the patient; an oxygen sensor for measuring an amount of oxygen provided to the patient via the fluid pump; a pulse oximeter for monitoring an oxygen saturation level of the patient; and a pump controller for controlling an amount of oxygen provided to the patient, via the fluid pump.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a front-side view of a portable breathing system according to embodiments of the present invention;

FIG. 2 is a perspective view of a left-side and a bottom portable of the portable breathing system shown in FIG. 1;

FIG. 3 is a perspective view of a right-side and the bottom of the portable breathing system shown in FIGS. 1-2;

FIG. 4 is a right-side view of the portable breathing system shown in FIGS. 1-3;

FIG. 5 is an exploded view of the portable breathing system shown in FIGS. 1-4;

FIG. 6 is a partial perspective view of an interior of the portable breathing system shown in FIGS. 1-5, with the portable breathing system including a fluid pump, a medical treatment connector, a T-connector, and an outlet;

FIG. 7 is a schematic depiction of a system for monitoring breathing system metrics according to embodiments of the present invention;

FIG. 8 is a flow chart of a method for regulating oxygen being supplied to a patient according to embodiments of the present invention; and

FIG. 9 is a flow chart of a method for monitoring breathing system metrics according to embodiments of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Embodiments of the present invention include an apparatus, computer program, method, and a system for providing pulmonary medical care to a patient, for obtaining a plurality of metrics relating to the breathing system and the patient, for wirelessly communicating the acquired information to one or more external computing devices, and for monitoring and analyzing the metrics via an external computing device. The computer program of embodiments of the present invention may include a primary computer program that is run on one or more external server devices and/or external computing devices and a secondary computer program that is run on a portable breathing system, each of which will be discussed in more detail below.

### Portable Breathing System

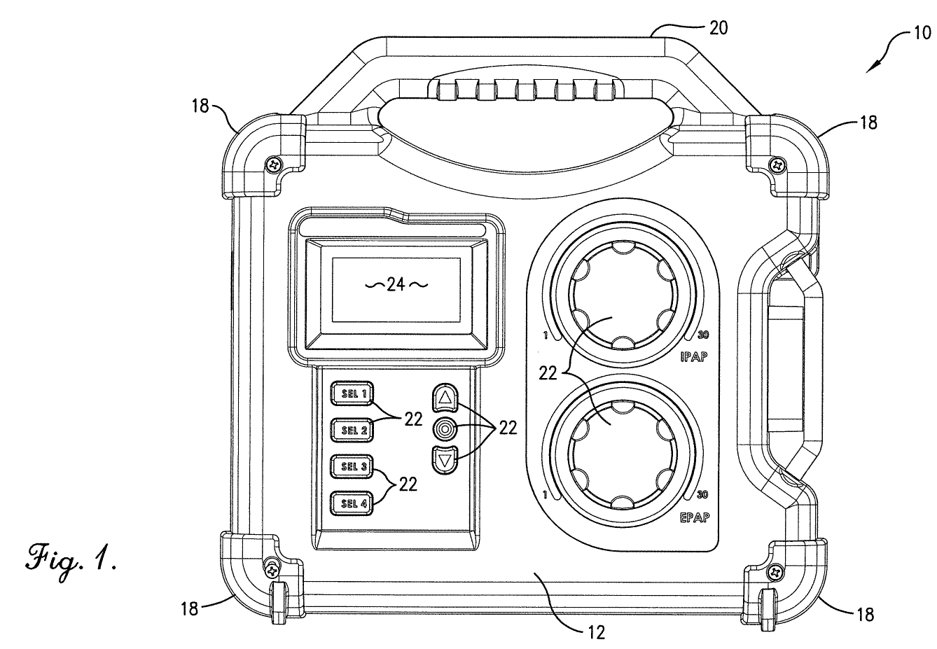

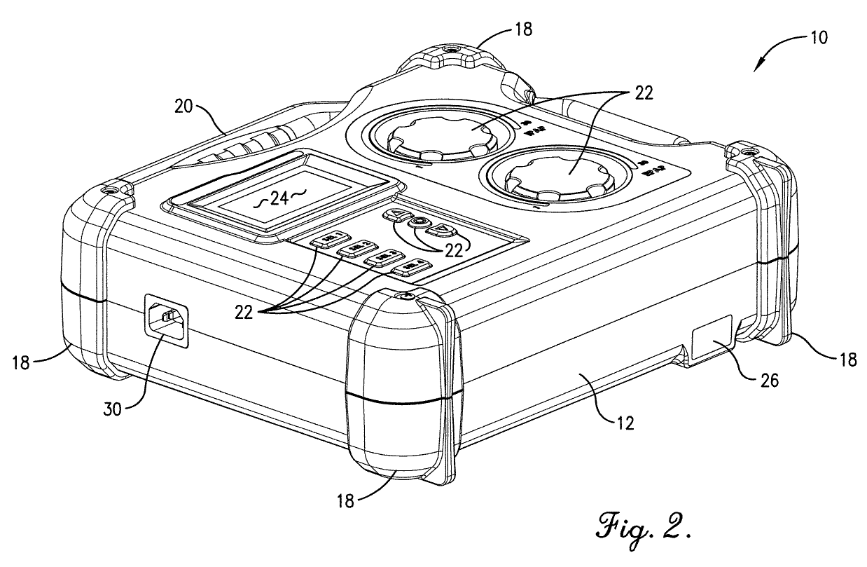

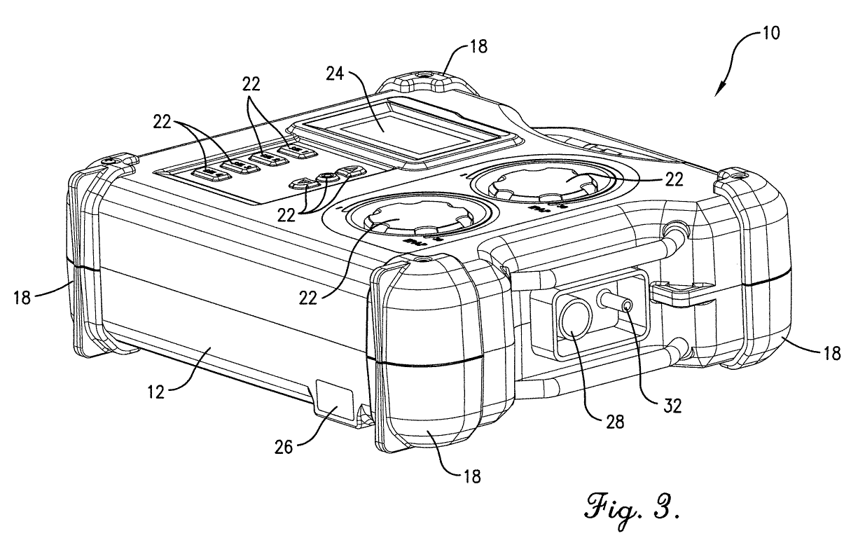

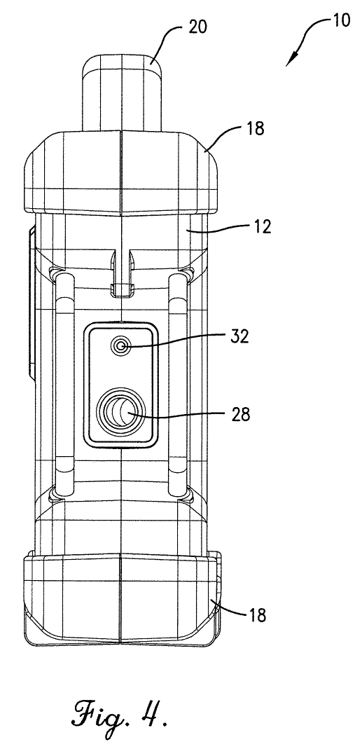

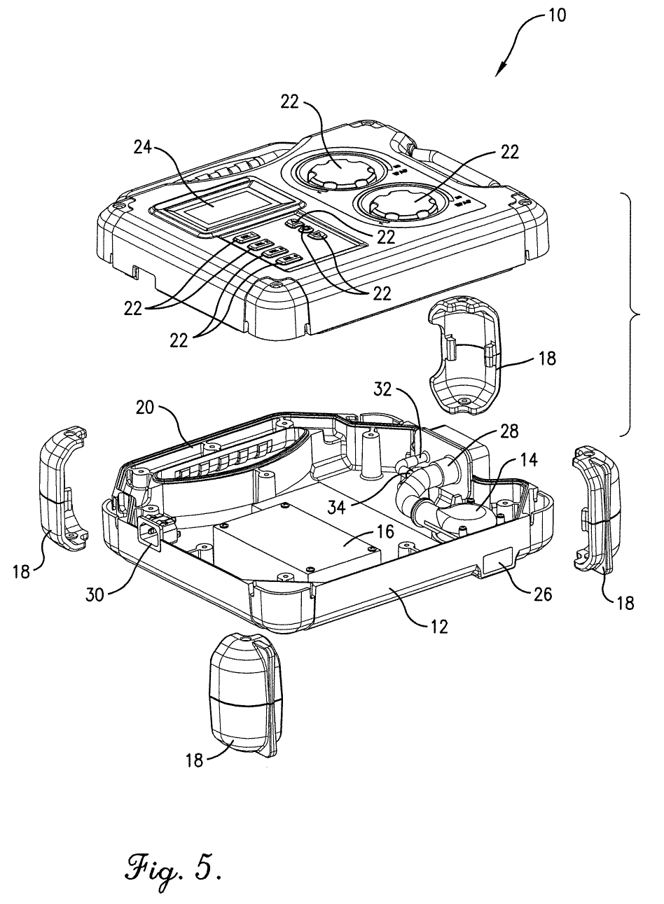

A portable breathing system 10, as shown in FIGS. 1-4, of embodiments of the present invention is directed to a system for providing positive airway pressure to a patient. With reference to FIG. 5, the portable breathing system 10 broadly comprises a housing 12; a fluid pump 14 positioned within the housing for providing airway pressure to the patient; one or more electronics modules 16 positioned within the housing, which may include one or more processing elements, memory elements, communications interface, a power source for providing electrical power to components of the breathing machine, and one or more sensors for sensing a plurality of breathing system metrics associated the portable breathing system and associated with a patient user using the breathing system. Embodiments of the present invention permit first-responder users to provide pulmonary medical care to patient users who may be unable to maintain clear airways, constant breathing, or oxygenation of blood. A first-responder user is an operator of the portable breathing system 10 who is providing treatment to the patient user. Embodiments of the present invention further provide for the sensed breathing system metrics to be communicated, via the communications interface, to an external computing device that is accessible by administrative users, such as doctors, nurses, and/or hospital staff, as discussed in detail below. Thus, embodiments of the present invention include a portable breathing system for providing positive airway pressure to a patient user, with the system being portable and including smart, interactive features. The portable breathing system 10 may be used by fire departments, ambulances, airlines, or in public buildings, airports, convention centers, or any other location where there may be a need to provide medical assistance to a patient that is having trouble maintaining an open airway or performing independent breathing.

The housing 12 of the portable breathing system 10 may preferably be sized to accommodate the remaining components of the system. For instance, embodiments of the present invention contemplate that the fluid pump 14, the one or more electronics modules 16 which includes the power source, the one or more sensors, and any additional components, which may be described in more detail below, may be located within the housing. As can be appreciated, a size of the housing 12 may vary depending on sizes of the components of the portable breathing system 10. For instance, for a portable breathing system that requires an extended operating time, the system may require a larger power source, such that the housing may be correspondingly larger to house the larger power source. Similarly, certain medical situations may require a portable breathing system with a larger fluid pump, such the housing may be correspondingly larger to house the larger fluid pump. Alternatively, if components of the portable breathing system are not required to be large in size, the size of the housing may correspondingly be reduced.

The housing 12 may be formed of ABS or other material suitable for rugged operation, such that the breathing machine can withstand exposure to high-level impacts, pressures, and temperatures. Returning to FIGS. 1-4, in certain embodiments, an exterior of the housing 12 may include rubber impact pads 18 to further protect the portable breathing system 10 from high-level impacts. The housing 12 may include a handle 20 attached to a top section of the portable breathing system 10, such as to facilitate portability. The exterior of the housing 12 may additionally include one or more user interface controls 22, such as dials, buttons, knobs, or the like, which may be used to control the function of the portable breathing system 10. The housing may also include an electronic display 24 that provides users with information related to the functionality of the portable breathing system 10 and/or the plurality of breathing system metrics obtained by the one or more sensors, as discussed in more detail below. In certain embodiments the electronic display 24 may include a liquid crystal display, cathode ray tube, light emitting diode, plasma display, or the like, which is capable of displaying graphics, text, images, videos, or the like to the first-responder users.

With reference to FIG. 5, the fluid pump 14 of the portable breathing system 10 may generally include any type of micro-air or gas pump that is known in the art. The pump may include a mechanical valve, rotary, diaphragm, solenoid, or other similar style of pump that is appropriate for applying positive airway pressure to a patient. The primary restriction on the fluid pump 14 is that it be sufficiently small in size and weight, so that the pump fits within the housing 12 and the portable breathing system 10 remains portable. The fluid pump 14 operates by drawing ambient air into the fluid pump via an inlet 26. In certain embodiments, the inlet 26 may include a filter element (not shown) that functions to filter or purify the ambient air from impurities before being drawn into the fluid pump 14. Once drawn in, the air is forced out of the fluid pump 14 and the housing 12 via an outlet 28. A flexible hose (not shown) may be connected to the outlet 28 at the flexible hose's proximal end. The flexible hose may be used to deliver the air from the pump to the patient user. The flexible hose may include a breathing mask (not shown) on its distal end. In certain embodiments, the breathing mask may fit over the patient user's mouth, forming a pressurized seal so that the pressure generated by the fluid pump 14 permits the introduction of air into the patient user's airway. The breathing mask may also include a mouthpiece that can be inserted into a patient user's mouth to keep the mouth open to receive air from the portable breathing system 10. In certain embodiments, the flexible hose and breathing mask may be carried in a detachable bag that may be attached to a bottom or back side of the portable breathing system 10.

The fluid pump 14 of embodiments of the present invention may provide two levels of adjustable air pressure to the patient user, hereinafter referred to as bi-level positive airway pressure (BPAP). A first level of air pressure, hereinafter inspiratory positive airway pressure (IPAP), provides a higher level of positive air pressure (i.e., higher than atmospheric air pressure) while the patient user is inhaling. A second level of air pressure, hereinafter expiratory positive airway pressure (EPAP), provides a lower level of positive air pressure (i.e., higher than atmospheric air pressure) while the patient user is exhaling. By maintaining a positive level of air pressure while the patient user is inhaling and exhaling, the fluid pump 14 of the portable breathing system ensures that the patient's airway remains open even if the patient is unable to inhale or exhale on her own. Thus, the portable breathing system 10 ensures that air will be introduced into the patient's lungs during each IPAP to EPAP cycle. In additional embodiments of the present invention, the fluid pump 14 may provide only a single fixed pressure, hereinafter continuous positive airway pressure (CPAP). Such CPAP functionality provides the patient user with a continuous positive air pressure that maintains the patient user's airway in an open position.

The electronics module 16 of the portable breathing system 10 may include various electrical components, such as one or more processing elements, memory elements, communications interfaces, power sources, and sensors. The electrical components of the electronics module 16 may be individual components, such that they are located and function independently of the other components. However, in other embodiments the electrical components of the electronics module 16 may be integrated as a single unit, such as on an integrated chip, printed circuit board, printed circuit assembly, or the like. The electronics module 16 may be held within the housing 16, or one more of the electrical component of the electronics module 16 may be located outside the housing.

The processing elements of the electronics module 16 of portable breathing system 10 generally execute a secondary computer program, wherein a computer program is also commonly known as instructions, commands, software code, executables, applications, apps, and the like. The processing elements may include processors, microprocessors, microcontrollers, field-programmable gate arrays (FPGAs), or the like, as well as combinations thereof. The secondary computer program associated with the processing element may include instructions that direct the operation of the portable breathing system 10, such as controlling the fluid pump 14 and receiving breathing system metrics from the one or more sensors. The secondary computer program may also instruct the processing elements to receive the plurality of metrics obtained from the one or more sensors and/or cause the plurality of metrics to be transmitted to an external computing device via the communications interface, as described in more detail below. In additional embodiments, the processing elements may perform calculations on the metrics received from the one or more sensors and may generate status information for the portable breathing system 10 or health information regarding the patient user. In certain embodiments, the processing elements may perform minimal processing actions on the metrics obtained from the one or more sensors. Instead, calculations and algorithms for analyzing the metrics and for reporting the same may be performed by an external computing device, such that the processing elements of the electronics module 16 may transmit the metrics to the external computing device for performing calculations. In additional embodiments, the portable breathing system 10 may write the metrics to the memory elements, discussed in more detail below, which can later and/or periodically be transferred to the external computing device for analysis. Thus, it should be appreciated that the secondary computer program stored on the memory elements in the portable breathing system may perform all or substantially all of the steps of embodiments of the present invention. However, it should also be appreciated that portions of the computer program could be stored on an external computing device, such that certain steps may be performed by the external computing device.

The memory elements of the electronics module 16 of portable breathing system 10 generally store the secondary computer program as well as the breathing system metrics received from the one or more sensors. The memory elements may further store other information such as data, text, graphics, videos, or other similar information, as may be necessary to carry out and perform embodiments of the present invention, as described herein. The memory elements may also be known as a “computer-readable storage medium” and may include non-transitory components such as random access memory (RAM), read only memory (ROM), flash drive memory, hard disk drives, or the like, as well as combinations thereof. The memory elements may be in electronic communication with the processing element, such that the processing element may access the secondary computer program associated with the processing element and data in the memory element in a manner known in the art. Certain embodiments may provide for the memory elements to be removable from the housing 12, such as with a secure digital (SD) cards, compact flash (CF) cards, writable Compact Disc, or the like. In some embodiments, a portion, or all, of the secondary computer program may be stored externally to the housing but may be accessible to the processing element through the communications interface, as will be discussed in more detail below.

The communications interface of the electronics module 16 generally allows the portable breathing system 10 to communicate with external devices, systems, computers, or networks, via a communications network discussed in more detail below. Generally, the communications interface includes a wireless connection, such as with radio frequency (RF) transmitters, receivers, or transceivers utilizing wireless communication protocols like GSM, CDMA, Bluetooth®, WiFi, WiMAX, or other radio or cellular protocols. In additional embodiments, the communications interface may have a wired connection, such as with electrically conductive cables, optical fibers, or the like. In embodiments that use the wired connection, the housing 12 of the portable breathing system 10 may include one or more universal connection ports, such as a Universal Serial Bus (USB), serial port, Ethernet, or FireWire connectors, to facilitate wired communications into the electronics module 16. Use of the communications interface allows information and data stored on the memory elements to be uploaded or otherwise transmitted to the external devices, systems, computers, or networks. Moreover, use of the communications interface allows software updates to be downloaded to the memory element for implementation of code segments of the secondary computer program associated with the processing element and stored on or associated with the memory elements of the portable breathing system 10. In additional embodiments, the communications interface may allow administrative users to communicate with first-responder users or patient users through the portable breathing system 10. The communication could be through voice, text, video, or other format as supported by the communications network. In even further embodiments, the communications interface may provide for administrative users to remotely control the functions and features of the portable breathing system 10, such as by remotely controlling the fluid pump 12, as will be described in more detail below.

The power source of the portable breathing system 10 may be stored in the housing 12 and functions to provide electrical power to the components of the system. In certain embodiments the power source may be a standard portable 12-Volt DC battery that is of sufficient size to fit within the housing 12. The housing 12 may include a recharging connector 30 that facilitates a recharging of the power source by a plurality of power systems such as AC, DC, solar, etc. In addition, the portable breathing system 10 may include a retractable power cord (not shown) that fits within the housing 12 and can extend out away from the housing to be plugged in to one or more of the plurality of power systems. The retractable power cord may power the components of the portable breathing system 10, charge the power source, or facilitate a power/recharge combination. For applications where extended, remote operation of the portable breathing system 10 is required, certain embodiments of the present invention provide for the power source to be interchangeable with back-up power sources. In such an embodiment, the power source included in the housing 12 may be removed and replaced with a back-up power source that is carried along with the portable breathing system 10. The back-up power source may be included and carried within the system's detachable bag, which was previously described.

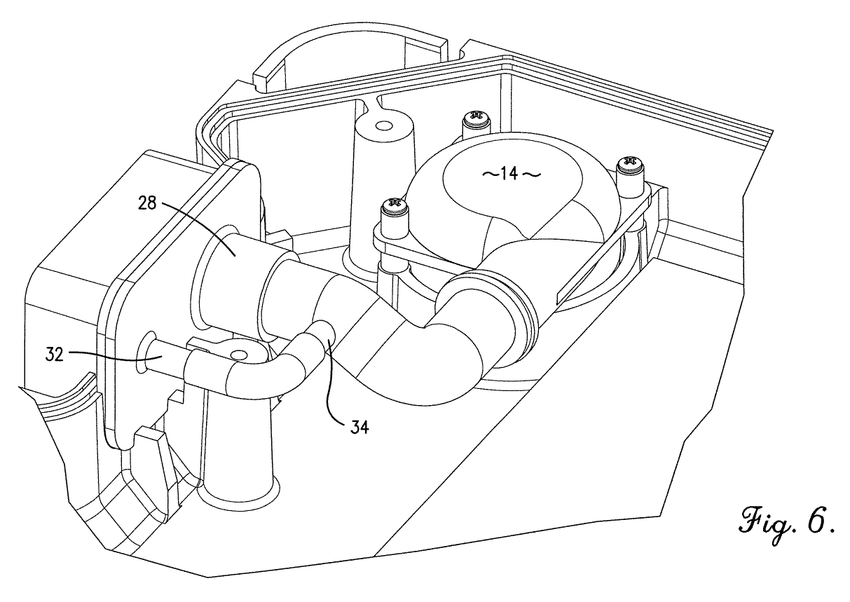

In certain embodiments of the present invention, the portable breathing system 10 may include additional medical treatment components that provide medications or other medically required substances to patient users. For example, the housing 12 may include a medical treatment connector 32 that is fluidly connected to the fluid pump 14, via a T-connector 34, such as illustrated in FIG. 6. The medical treatment connector 32 may permit connection to an oxygen source (e.g., an oxygen tank) or a humidity source (e.g., a water reservoir). Therefrom, the air that is provided to the user from the fluid pump 14 may be mixed with the oxygen or the water, such that oxygen rich or humidified air may be delivered to the patient user. Other medical treatment components may be attached to the housing 12, via the medical treatment connector 32, to facilitate the introduction of aerosol-based medications to the patient. Once the aerosol-based medications are introduced to the pump, the medications may then be forced through the flexible hose and mouthpiece before being introduced to the airway of the patient user. Certain of the above-mentioned additional components may be carried in the detachable bag that is attached to the bottom or back of the machine. The portable breathing system 10 may include sensors that can monitor metrics associated with the introduction of medications or other medially required substances to the patient user, such as fluid flow rate sensors, oxygen sensors, medication sensors, humidity sensors, and air filter sensors. Such sensors may be positioned within the portable breathing system 10, for instance, within the fluid pump 14, the outlet 28, the medical treatment connector 32, the T-connector 34, or other locations where it may be necessary to sense such metrics. In addition, the sensors may provide critical information such as the level of oxygen remaining in the oxygen tank, the level of water remaining in the water tank reservoir, the amount of medication remaining, or the amount of operational time left for the filter. Each of the sensors discussed above are in electrical communications, either hard-wired or wirelessly, with the electronics module 16.

As previously described, the portable breathing system 10 may include one or more sensors for sensing one or more breathing system metrics associated with component of the portable breathing system or associated with the patient user using the portable breathing system. Some of the sensors may be located within the housing, while other sensors may be positioned and/or employed outside of the housing. Sensors located within the housing 12 may communicate with other components of the electronics module 16 by a direct electrical connection. For those sensors that are employed outside of the housing, the sensors may be attached to the breathing machine through one of the one or more universal connection ports, such that the sensed signals can be transmitted from the sensor to the memory elements and/or processing elements of the electronics module 16. In additional embodiments, the external sensors may transmit the breathing system metrics to the electronics module 16 wirelessly, through the communications network. Further, certain breathing system metrics may be sensed directly by components of the portable breathing system 10, such that individual or separate sensors are not required to obtain such metrics. As an example, the fluid pump 14 may have internal sensor integrated inside the fluid pump, such that operating parameters, flow rates, pressures, or the like may be monitored directly by the fluid pump and communicated to the electronics module 16.

Exemplary breathing system metrics that may be sensed may include metrics associated with the portable breathing system 10, such as an operating mode of the fluid pump 12 (i.e., BPAP or CPAP), an IPAP value, an EPAP value, inspiratory and expiratory times, IPAP/EPAP ratio, amount of oxygen provided to the patient user, amount of humidity provided to the patient, amount of other medications provided to the patient, or other similar metrics. Such metrics may be directly sensed by the fluid pump 14 or by sensors positioned within the portable breathing system 10 and configured to sense such metrics. Additional metrics associated with the portable breathing system 10 that may be sensed may include a remaining charge of the power source, a remaining level of oxygen in the oxygen source, a remaining level of water in the humidity source, a remaining amount of medication in the medication source, the remaining filter life of the filter, or the like. Similarly, such metrics may be directly sensed by the components of the breathing system 10 or by sensors positioned within the system and configured to sense such metrics. The metrics that may be sensed may also include environmental information such as ambient temperature, ambient humidity, and/or ambient pressure. Thus, embodiments of the present invention may include the necessary sensors required to collect the information related to the metrics provided above (i.e., thermometers, hygrometers, barometers, etc.). In even further embodiments, time stamps associated with each metric may be obtained, such that the metrics may be analyzed with respect to time (e.g., time rates of change).

Additional embodiments of the present invention may provide for the portable breathing system 10 to include an impact sensor, such as an accelerometer or g-shock sensor, which can sense if the breathing system has been exposed to a high-impact event. The portable breathing system 10 may also be included with a global positioning system (GPS) sensor, such that the position and movement of the machine can be monitored and tracked. Embodiments of the present invention further provide for power source sensors that function to sense metrics associated with the power source. Exemplary power source metrics may include a voltage, a current, an impedance, a temperature, or the like. Such power source metrics may be used to analyze status information of the power source, such as state of charge or state of health.

In addition to breathing system metrics associated with the portable breathing system 10, embodiments of the present invention may include sensors for obtaining breathing system metrics associated with the patient user. For instance, such metrics may include the patient user's breathing rate, breathing tidal volume, body temperature, air temperature entering and leaving the patient user, pulse, blood pressure, blood oxygen level, carbon dioxide levels, or the like. Embodiments of the present invention may include specific sensors necessary to obtain the above-described metrics, such as fluid flow rate sensors, thermometers, oxygen sensor, carbon dioxide sensor, pulse oximeter, sphygmomanometer, heart monitor, or the like.

### Monitoring and Control System

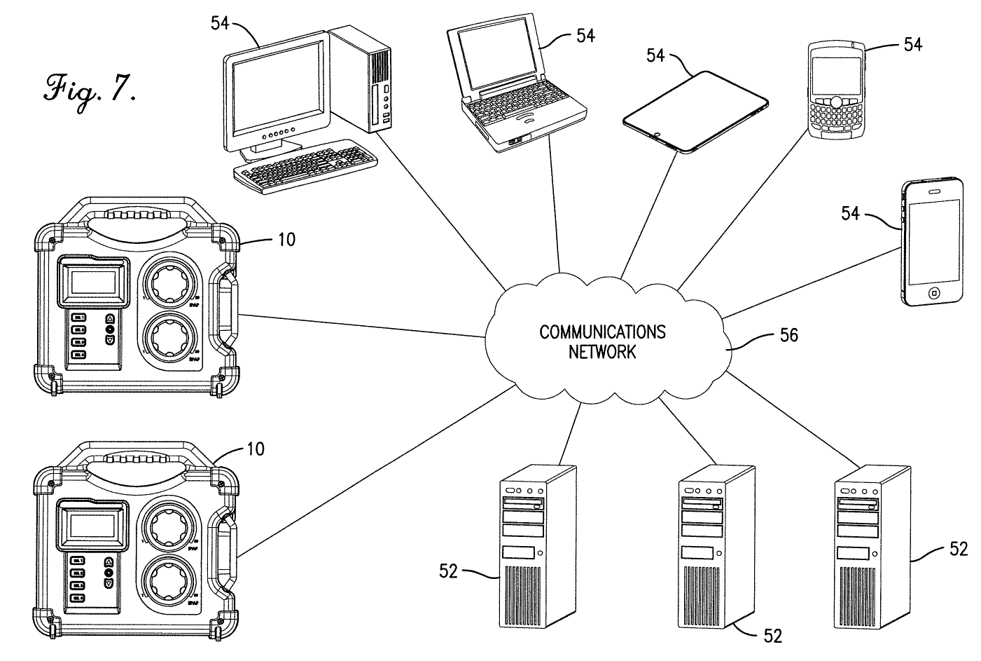

As described above, embodiments of the present invention may additionally provide for breathing system metrics to be provided and manipulated by external computing devices. In even further embodiments, the external computing devices may be used to control the functionality of the portable breathing system 10. Such embodiments of the present invention may be implemented in hardware, software, firmware, or combinations thereof using monitoring and control system 50, shown in FIG. 7, which broadly comprises server devices 52, external computing devices 54, a communications network 56, and one or more portable breathing systems 10, as were previously described. The server devices 52 may include computing devices that provide access to one or more general computing resources, such as Internet services, electronic mail services, data transfer services, and the like. The server devices 52 may also provide access to a database that stores information and data necessary for the implementation of a primary computer program, method, and other embodiments of the present invention. The primary computer program and computing devices illustrated and described herein are merely examples of programs and computing devices that may be used to implement aspects of embodiments of the invention and may be replaced with other programs and computing devices without departing from the scope of the invention.

The server devices 52 and external computing devices 54 may include any device, component, or equipment with a processing element and associated memory elements. The processing element may implement operating systems, and may be capable of executing a primary computer program, which is also generally known as instructions, commands, software code, executables, applications (“apps”), and the like. The processing element may include processors, microprocessors, microcontrollers, field programmable gate arrays, and the like, or combinations thereof. The memory elements may be capable of storing or retaining the primary computer program and may also store data, typically binary data, including text, databases, graphics, audio, video, combinations thereof, and the like. The memory elements may also be known as a “computer-readable storage medium” and may include random access memory (RAM), read only memory (ROM), flash drive memory, floppy disks, hard disk drives, optical storage media such as compact discs (CDs or CDROMs), digital video disc (DVD), Blu-Ray™, and the like, or combinations thereof. In addition to these memory elements, the server devices 52 may further include file stores comprising a plurality of hard disk drives, network attached storage, or a separate storage network. The functionality of server devices 52 may also be distributed amongst many different computers in a cloud computing environment.

At least one of the server devices 52 may operate and/or host a website accessible by at least some of the external computing devices 54. The server device 52 may include conventional web hosting operating software, an Internet connection, such as a cable connection, satellite connection, DSL converter, or ISDN converter, and is assigned a URL and corresponding domain name so that the website hosted thereon can be accessed via the Internet in a conventional manner. In embodiments of the invention where the server device 52 implements a mobile application (i.e., an “app”), the server device may host and support software and services of proprietary mobile application providers, such as Google, Apple, and Blackberry. For example, some server devices 52 may support Google Android mobile applications, while other server devices may support Apple iPhone mobile applications.

The external computing devices 54 may specifically include mobile communication devices (including wireless devices), work stations, desktop computers, laptop computers, palmtop computers, tablet computers, portable digital assistants (PDA), smart phones, and the like, or combinations thereof. Various embodiments of the external computing device 54 may also include voice communication devices, such as cellular and/or mobile phones. In preferred embodiments, the external computing device 54 will have an electronic display, such as a cathode ray tube, liquid crystal display, plasma, or touch screen that is operable to display visual graphics, images, text, etc. In certain embodiments, the secondary computer program of the present invention facilitates interaction and communication through a graphical user interface (GUI) that is displayed via the electronic display. The GUI enables the user to interact with the electronic display by touching or pointing at display areas to provide information to the user control interface, which is discussed in more detail below. In additional preferred embodiments, the external computing device 54 may include an optical device such as a digital camera, video camera, optical scanner, or the like, such that the computing device can capture, store, and transmit digital images and/or videos. The external computing devices 54 may include a user control interface that enables one or more users to share information and commands with the computing devices or server devices 52. The user interface may facilitate interaction through the GUI described above or may additionally comprise one or more functionable inputs such as buttons, keyboard, switches, scrolls wheels, voice recognition elements such as a microphone, pointing devices such as mice, touchpads, tracking balls, styluses.

The communications network 56 of the system 50 may be the same communications network described with respect to the portable breathing system 10. The communications network may also be a combination of several networks. For example, the external computing devices 54 may wirelessly communicate with a another external computing device 54 or a server 52 in a building or facility via a Wi-Fi network, which in turn is in communication with one or more of the servers 52 or portable breathing systems 10 via the Internet, cellular network, or other communications network.

Both the server devices 52 and the computing devices 54 may be connected to the communications network 56. Server devices 52 may communicate with other server devices 52, external computing devices 54, and/or the one or more portable breathing systems 10 through the communications network 56. Likewise, external computing devices 54 may communicate with other external computing devices 54, server devices 52, and/or the one or more portable breathing systems 10 through the communications network 56. Thus, the server devices 52 and the external computing devices 54 may include the appropriate components to establish a connection with the communications network 56.

The primary computer program of the present invention may run on external computing devices 54 or more server devices 52. In alternative embodiments, the primary computer program may run on one or more portable breathing systems 10. Additionally, a first portion of the program, code, or instructions may execute on a first server device 52 or first computing device 54, while a second portion of the program, code, or instructions may execute on a second server device 52 or a second computing device 54. In some embodiments, other portions of the program, code, or instructions may execute on other server devices 52 or external computing devices 54 as well. For example, information and data may be stored on a memory element associated with the server device 52, such that the information and data is remotely accessible to users of the primary computer program via one or more external computing devices 54. Alternatively, the information and data may be directly stored on the memory element associated with the one or more external computing devices 54. In additional embodiments of the present invention, a portion of the information and data may be stored on the server device 52, while another portion may be stored on the one or more external computing devices 54. The various actions and calculations described herein as being performed by or using the primary or secondary computer program may actually be performed by one or more computers, processors, or other computational devices, such as the computing devices 54, server devices 52, or portable breathing systems 10 independently or cooperatively executing portions of the primary and secondary computer program.

In certain embodiments of the present invention, the primary computer program may be embodied in a stand-alone program downloaded on a user's computing device 54 or in a web-accessible program that is accessible by the user's computing device 54 via the communications network 56. The executable form of the program permits the user to access embodiments of the present invention via an electronic resource, such as a mobile “app” or website. For the stand-alone program, a downloadable version of the secondary computer program may be stored, at least in part, on the server device 52. A user may download at least a portion of the secondary computer program onto the computing device 54 via the network 56. In such embodiments of the present invention, the primary computer program may be implemented as an “application,” such as an “app” for a mobile device. After the primary computer program has been downloaded, the program can be installed on the computing device 54 in an executable format. For the web-accessible computer program, the user may simply access the computer program via the network 56 (e.g., the Internet) with the external computing device 54.

Once a user has access to the electronic resource, via the secondary computer program installed on a user's external computing device 54 or the web, certain embodiments may provide for users to create user accounts with which to access the electronic resource. The user accounts may be stored within the memory elements of the external computing device 54, the server device 52, or in the associated database. Certain embodiments of the present invention may provide for at least two types of accounts: a medical user account and an administrative user account, hereinafter referred to as an admin account. Each type of user account may provide their respective users with unique roles, capabilities, and permissions with respect to implementing embodiments of the present invention. Each account may be associated with a username and/or password required to be entered by a particular user prior to accessing the account. In addition, embodiments of the present invention may include any number and/or any specific types of account as may be necessary to carry out the functions, features, and/or implementations of the present invention.

A medical user account is an account created by or for medical users, such as nurses, doctors, medical staff, or others who may implement embodiments of the present invention to receive and analyze breathing system metrics obtained from one or more portable breathing systems 10. In addition, the medical user account may be used by medical users to control the functions and features of one or more portable breathing systems 10, as will be discussed in more detail below. Admin account is an account created by or for administrative users, such as medical administrators, managers, and staff, who may be responsible for administrating embodiments of the present invention. For instance, the admin account may be used to create and maintain user accounts and/or to generally oversee embodiments of the present invention. Each user with a user account may be required to enter certain identification information that is associated with the user account, such as name, workplace title, email address, telephone number, or the like. In certain embodiments and as discussed in more detail below, the email address and telephone number associated with the user account may be used to receive alerts and notifications associated with the portable breathing system 10 or a patient user thereof.

### Operation

The portable breathing system 10 of embodiments of the present invention functions to keep a patient user's airway open by providing positive pressure to the patient's airway. As previously described, the portable breathing system 10 may include a flexible hose and breathing mask attached to the outlet 28. Therefrom, the fluid pump 14 of the portable breathing system 10 functions to provide positive airway pressure in either a BPAP or CPAP modes, as previously described. A first-responder user can vary the mode of the portable breathing system 10 by manipulating the user interface controls 22 (i.e., the buttons, knobs, dials). The selected mode may be displayed as a metric to the first-responder via the electronic display 24. If the BPAP mode is selected, embodiments of the present invention additionally provide for the first-responder user to vary the IPAP and/or EPAP levels, the time between IPAP to EPAP cycles, IPAP/EPAP ratios, or other functionality through manipulation of the user interface controls 22 on the housing 12. Such settings and/or changes may be displayed as breathing system metrics on the electronic display 26. For instance, with reference to FIG. 1, the first-responder user may rotate the circular dials of the user interface controls 22 to manually adjust each of the IPAP and EPAP pressure levels. Similarly, if the CPAP mode is selected, the first-responder user can select the air pressure level supplied to the patient user by manipulating the user interface controls 22. When the air pressure has been selected, the selected pressure levels may be displayed as a metric on electronic display 24.

Embodiments of the present invention further provide for first-responder users to control amounts of the additional medications or other medically required substances that are provided to the patient user by manipulating the user interface controls 22. For instance, the first-responder user can manually set an amount of oxygen being supplied to the patient user via the oxygen source by selecting an appropriate amount via the user interface controls 22. Similarly, the first-responder user can adjust the amount of humidity and/or other medications that are provided to the patient user. The amount of the oxygen, humidity, medications, or other medically required substances being administered to the patient user may be displayed on the electronic display 24. Such amounts may be determined via the associated sensors and/or medical treatment components included in the portable breathing system 10. In certain other embodiments, the amount of medications may be controlled by directly manipulating the additional medical treatment components, such as the oxygen source, humidity source, and/or medication source. However, in such embodiments, the electronic display of embodiments of the present invention may continue to display the amounts of medications being provided to the patient user.

Embodiments of the present invention further provide for additional breathing system metrics obtained by the portable breathing system 10 to be displayed via the electronic display 24. For instance, the remaining charge of the power source, the remaining level of oxygen in the oxygen source, the remaining level of water in the humidity source, the remaining amount of medication in the medication source, and/or the remaining filter life of the filter may each be displayed. In addition, if the portable breathing system 10 has been exposed to a high-impact event, as indicated by the high-impact sensor, an alert may be presented on the electronic display 24, or alternatively presented through an audible alert. Such an alert may indicate to the first-responder user that the portable breathing system was exposed to a high-impact event and may be in need of maintenance. In even further embodiments, the GPS indicated location of the portable breathing machine 10 may be displayed on the electronic display. The electronics module may also be configured to determine a distance to a medical facility and amount of time required to reach the medical facility. Such information may be presented on the electronic display 24, such that the first-responder use may judge how long the patient-user will be required to use the portable breathing system 10 until they reach the medical facility. The electronic display may also display other breathing system metrics, such as ambient temperature, ambient humidity, ambient pressure, or the like.

In addition to the metrics associated with the portable breathing system 10, as discussed above, embodiments of the present invention provide for the breathing system metrics associated with the patient user to be displayed on the electronic display 24. For instance, the patient user's breathing rate, breathing tidal volume, body temperature, air temperature, blood pressure, blood oxygen level, carbon dioxide level, or other similar metrics may be displayed on the electronic display 24 for viewing and use by the first-responder user. In addition, should any metrics associated with the portable breathing system 10 or the patient user fall below a predefined threshold, the portable breathing system may present a notification or an alert to the first-responder user. Such predefined threshold may be set by the first-responder user or other users of embodiments of the present invention. For example, if the patient user's oxygen level drops below a predefined threshold, the portable breathing system 10 may emit an audible alert and provide a description of the notification on the electronic display 24. In additional embodiments, the power source metrics may be displayed on the electronic display 24, or alternatively, on a secondary display located on the housing 12 of the portable breathing system 10.

In even further embodiments, first-responder users may be presented with videos on the electronic display 24 of the portable breathing system 10. In such an embodiment, the electronic display 24 may, for instance, display instructional videos to the first-responder user. For example, the instructional videos may include instructional information as to the portable breathing system 10 operation, medical techniques and guidelines, or other general information as may be required to operate the portable breathing system or to treat the patient user. Videos may be stored on the memory elements of the portable breathing system 10 or may be streamed live from the server devices 52 over the communications network 56.

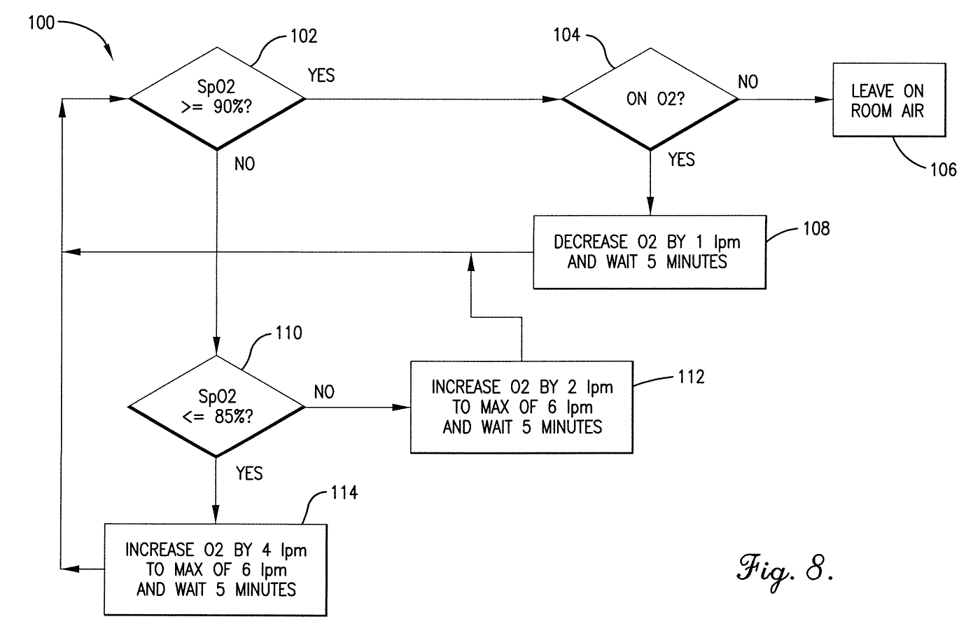

As previously described, the first-responder user can manually adjust the medication amounts being administered to the patient user. Embodiments of the present invention may be used to provide precise medication amounts as may be required for specific medical protocols. For instance, an exemplary oxygen titration protocol method 100 is illustrated in FIG. 8. The oxygen titration protocol indicates how much oxygen should be administered to a patient user that is undergoing a pulmonary medical issue. Beginning with Step 102, and with an airflow being provided to a patient user, embodiments of the present invention provide for the first-responder user to observe the patient user's oxygen level (i.e., blood oxygen saturation level). Such an oxygen level may be a metric that is obtained by, for instance, a pulse oximeter attached to the patient user and electronically connected to the portable breathing system 10. The oxygen level may be displayed via the electronic display 24. If the patient user's oxygen level is above 90 percent, then in Step 104, the first-responder user may determine whether external oxygen from the oxygen source is being administered to the patient user. As with the oxygen level, an indication of whether oxygen is being provided and an amount of oxygen being provided can be obtained via sensors and displayed on the electronic display 24. For instance, the portable breathing system 10 may include a flow rate sensor connected to the oxygen source, the medical treatment connector 32, or the T-connector 34, to sense the amount of external oxygen being supplied from the oxygen source. In additional embodiments, the portable breathing system 10 may include additional flow rate sensors on the fluid pump 14, the T-connector 34, or the outlet 26, to sense an amount of oxygen being supplied from the fluid pump (i.e., oxygen from the ambient air).

If external oxygen from the oxygen source is not being provided, then the first responder user permits the portable breathing system 10 to continue to provide airflow to the patient user only from the fluid pump 14, as in Step 106, without mixing any external oxygen. If in Step 104, external oxygen was being mixed with the airflow and provided to the patient user, then in Step 108, the amount of external oxygen may be reduced, by approximately 1 liter per minute. As previously noted, the amount of external oxygen being provided may be displayed on the electronic display, such that the first-responder user can determine the amount being administered. Additionally, the first-responder user may adjust the amount of external oxygen being administered by manipulating the user interface controls 22. Once the amount of external oxygen being provided has been appropriately reduced, the first-responder user waits five minutes and then returns to Step 102.

Remaining with the method 100 illustrated in FIG. 8, if in Step 102, the patient user's oxygen level is under 90 percent, the first-responder user may then transition to Step 110 and observe whether the patient user's oxygen level is above or below 85 percent. If the patient user's oxygen level is above 85 percent, then, in Step 112, the first-responder user can increase the amount of external oxygen being supplied to the patient user by 2 liters per minute (up to a maximum of 6 liters per minute) and return to Step 102. After waiting five minutes, Step 102 may be repeated. If at Step 110, however, the patient user's oxygen level is below 85 percent, then, in Step 114, the first-responder user can increase the amount of external oxygen being supplied to the patient user by 4 liters per minute (up to a maximum of 6 liters per minute) and return to Step 102. After waiting five minutes, Step 102 may be repeated.

As described above, the amount of external oxygen being supplied to the patient user may be determined by sensors positioned in the portable breathing system 10, which determine a percentage of oxygen included in the airflow being provided to the patient user. In additional embodiments, the sensor may be positioned externally from the portable breathing system, such as within or attached to the oxygen source. The steps included in method 100 may thus provide for the display and the adjustment of oxygen levels based on the percentage of oxygen contained in the airflow. In additional embodiments of the present invention, the first-responder user may preset an oxygen amount (e.g., by liters per minute or by percentage) to be provided to the patient user, and the portable breathing system 10 may automatically determine the amount of oxygen being provided and adjust such amount to maintain the preset amount.

In even further embodiments, the method 100 may be performed in an automated fashion by the portable breathing system 10. For instance, the method 100 may be performed via the second computer program and processing element of the portable breathing system 10 by the system monitoring the patient user's oxygen level as determined by the one or more sensors (e.g., the pulse oximeter) and the amount of external oxygen being supplied to the patient user as determined by the one or more sensors. Based on the patient user's blood oxygen level, the portable breathing system 10 may automatically adjust the oxygen amount provided to the patient user according to Steps 102-114. In such embodiments, the portable breathing system may include an electrically controlled valve that functions to control a flow rate of external oxygen from the oxygen source to the outlet 28. The electrically controlled valve may be positioned at the oxygen source, the medical treatment connector 32, or at the T-connector 34. The electrically controlled valve may be controlled by the processing element and/or controller of the electronics module 16, via a feedback circuit connected to each of the sensors (e.g., oximeter and/or flow rate sensors) and the electrically controlled valve. For instance, if the patient user's oxygen level is below between 90 and 85 percent, then the portable breathing system 10 may automatically increase the amount of supplied external oxygen by 2 liters per minute, as required in Step 110. After waiting five minutes, which may be indicated by an internal timer included in the electronics module 16, the portable breathing system 10 may again determine the patient user's oxygen level and perform the required step. Thus, the portable breathing system 10 may independently and automatedly perform each of the steps included in method 100.

In addition to automatedly controlling the amount of oxygen provided to a patient user, the portable breathing system 10 may automatedly control other functions and features. For instance, the portable breathing system 10 may automatedly control provided medication amounts, the BPAP or CPAP modes, the IPAP and/or EPAP levels, the time between IPAP to EPAP cycles, IPAP/EPAP ratios, etc. Such functions and features may be set to particular values by a user, and the portable breathing system 10 may automatically adjust the functions and features to maintain the particular values. Alternatively, the portable breathing system 10 may automatedly adjust the functions and features based on other sensed breathing system metrics. For instance, if the patient's user's oxygen level begins to drop, the portable breathing system 10 may automatically increase the IPAP and/or EPAP levels or decrease the time between IPAP to EPAP cycles.

In addition to presenting breathing system metrics directly on the electronic display 24 of the portable breathing system 10, one or more portable breathing systems 10 may transmit metrics to one or more server devices 52 included in a system 50. The server devices 52 may store such received metrics in the memory elements and/or associated databases for organizational and referencing purposes. Such purposes may include monitoring and analyzing statuses or operational histories of the one or more breathing systems 10. The transmission of the metrics from the portable breathing systems 10 may be performed wirelessly, via the communications interface, in real-time. In additional embodiments, the transmission of metrics may be performed at desired intervals, such as every hour, every thirty minutes, every minute, nearly instantaneously, etc. Embodiments of the present invention may further allow for users to select a frequency for sending and receiving updated metric information.

The external computing devices 54 of the system 50 may be used by medical user or administrative users who are required or who desire to review the breathing system metrics, breathing system status information, patient user health information, and/or operational histories for one or more portable breathing systems 10. Embodiments of the present invention provide for each of the one or more portable breathing systems 10 to have an individually associated electronic resource, such as a website or app, which can provide medical and administrative users with real-time breathing system metrics, as sensed and transmitted by each of the one or more portable breathing system 10. The electronic resource may thus present users with real-time breathing system metrics as transmitted by each portable breathing system 10. Embodiments of the present invention may further perform analyses on breathing system metrics associated with the portable breathing system 10 to provide users with status information of the portable breathing system. The status information may include any of the breathing system metrics and may further include other various information obtain from the metrics, such as operating conditions of the portable breathing system 10, time rates of change of the metrics, metric comparisons, metric projections, or the like. The breathing system metrics may also include metrics associated with the patient user. Embodiments of the present invention may further perform analyses on such breathing system metrics associated with the patient user to provide users with health information on the patient user. The health information may include any of the breathing system metrics and may further include other various forms of information obtained from the metrics, such as comparison of such metrics with normalized metrics, time rates of change of the metrics, metric projections, or the like. Embodiments of the present invention may further provide for the generation of operational histories, which may include any historical data related to breathing system metrics, status information, and/or health information. Thus, such metrics, status information, health information, and operational histories may be presented in various forms, such as by numerical values, graphs, charts, tables, or the like. The electronic resource may provide multiple individual metrics, status information, health information, or operating histories on a single screen of the electronic resource, via multiple panes. The electronic resource may additionally, or alternatively, be configured to include a summary screen that provides summary information, such as a summary of all metrics, status information, health information, and operating histories, for any or all of the portable breathings systems 10 included in the system 50. Alternatively, the electronic resource may be used to view metrics obtained from a single portable breathing system 10.

Embodiments of the present invention may provide for the creation of a customizable electronic resource where medical and/or admin users can personalize the presentation of breathing system metrics, status information, and/or operational histories from each of the one or portable breathing machines 10 in the system 50. Such electronic resource may include a summary screen that provides summary information for all breathing system metrics obtained and transmitted by each portable breathing system 10, including a summary of each portable breathing system 10 currently in use and of each patient user currently being treated. The electronic resource may also be used to view breathing system metrics obtained from a single portable breathing system 10. The electronic resource may additionally provide the users with alerts or notifications that inform the users as to the status of a particular breathing system 10 or of a particular patient user, including any of the breathing system metrics collected by the one or more sensors. For example, a medical user, who is a nurse, may be viewing an electronic resource associated with a portable breathing system that is currently treating a patient user who is experiencing chest pains. By observing certain metrics (e.g., breathing rate, breathing volume, blood pressure, oxygen level) collected by the portable breathing system 10, the medical user may make special preparations to properly equip the medical facility with treatment equipment based on the observed metrics. The users may also implement notification features that actively inform the users, in real-time, as to certain breathing system metrics. The users may also input certain predefined threshold values, such that if any of the breathing system metrics violate the threshold values, an alert may be sent to the user. For instance, if the patient user's oxygen level drops rapidly, the system 50 may provide for the medical user's external computing device 54 to receive an alert, such as a page, text, email, or the like, which indicates that the medical user should be preparing for a medical emergency upon arrival of the patient user. Such notifications or alerts may be sent via the user's email address and/or telephone number that is associated with the user's user account. Additionally, such notification and/or alert may be presented via an video/audio alert on the user's external computing device 54. All of the breathing system metrics associated with the patient may be saved and recorded in the sever devices 52 or the associated database for medical recordkeeping purposes. Such metrics may also be added to the patient user's medical charts and files. In even further embodiments, the medical users may use the external computing devices 54 to display the current location of the one or more portable breathing systems 10 and/or to determine and display the amount of time it will take for the portable breathing system to reach the medical facility. Thus, the medical user can determine how much time will be available to make any necessary preparations for receiving the patient user.

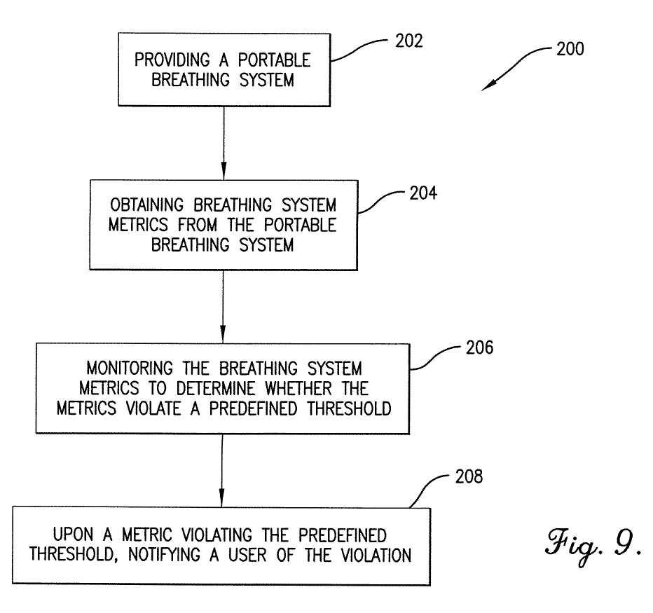

Thus, embodiments of the present invention include a method 200, as illustrated in FIG. 9, for monitoring breathing system metrics obtained from a portable breathing system. The method 200 includes the initial Step 202 of providing a portable breathing system that includes a fluid pump; a controller for controlling the fluid pump; and a communications interface for wirelessly transmitting the breathing system metrics. In Step 204, the breathing system metrics obtained from the portable breathing system may be received. Next, in Step 206, the breathing system metrics can be monitored to determine whether any of the breathing system metrics violate a predefined threshold. Finally, in Step 208, if a metric falls below the predefined threshold, a notification can be sent to a user, notifying the user of the metric falling below the threshold.

In additional embodiments, administrative users, such as hospital managers and administrative personnel, may have their own administrative electronic resource were they may view breathing system metrics related to certain logistic information of the portable breathing system 10, such as a status of the portable breathing system, a power level of the power source, the filter level of the air filter, or levels of the medical treatment components. For instance, through the electronic resource, the administrative user could receive a notification that the portable breathing system's air filter is nearing the end of its useful life, and thus a new air filter should be added to the system as soon as practical. The administrative users may have the option to add notification features that could actively inform the users, in real-time, as to certain breathing system metrics. For instance, if the breathing system metrics indicate that the power source of the portable breathing system 10 is failing and will not maintain an electrical charge, the system 50 may provide for the administrative user's external computing device 54 to receive an alert, such as a page, text, email, or the like, which indicates that the portable breathing system's power source requires immediate maintenance. Additional features directed to obtaining breathing system metrics associated with the power source are disclosed in U.S. patent application Ser. No. 13/832,307 filed Mar. 15, 2013, entitled APPARATUS, COMPUTER PROGRAM, METHOD, AND SYSTEM FOR ACQUIRING AND ANALYZING BATTERY METRICS, which is hereby incorporated by reference in its entirety.

In even further embodiments of the present invention, the administrative user may be able to prepare a variety of status reports regarding any or all of the breathing system metrics by providing charts, graphs, or other desired information for future use, review, and documentation. The status reports may be helpful for legal recordkeeping and for reducing insurance costs. Such status reports may also be used to quickly and easily determine which portable breathing systems 10 need maintenance, which is especially helpful for those entities have numerous portable breathing systems. The administrative user may also prepare custom status reports for each of the one or more portable breathing systems 10. The administrative user may select preferred delivery method(s) and generation frequency of status reports. For instance, the status reports may be delivered to the administrative user at desired intervals, such as daily, weekly, monthly, or the like. These status reports may be stored by the sever device 52 or the external computing device 54 so as to create a history or log of portable breathing system 10 performance. The generation of status reports may be performed without user input, thus allowing administrative users to forgo the continual manual monitoring of the breathing system metrics. Embodiments of the present invention may also notify the administrative or first-responder users when it is time to replace various components of the portable breathing system 10. For example, a user may be alerted when it is time to replace the power source or when it is time to replace the air filter. The administrative users may also perform additional administrative tasks via embodiments of the present invention, such as registration of additional electronic resources and/or portable breathing systems 10 to the monitoring and control system 50.

In certain other embodiments, the external computing devices 54 may be used by medical users to remotely control, in real-time, the functions and features of one or more portable breathing systems 10. The medical users may control any of the functions and features, as described here, such as provided medication amounts, the BPAP or CPAP modes, the IPAP and/or EPAP levels, the time between IPAP to EPAP cycles, IPAP/EPAP ratios, etc. For example, a doctor who is monitoring, via an external computing device 54, the breathing system metrics associated with a patient user of a portable breathing system 10 may observe that the patient user's breathing rate is too low. In response, the doctor may remotely increase the breathing rate as administered by the portable breathing system directly from the external computing device 54. Similarly, the doctor may control the amount of external oxygen being provided to the patient user, such as according to method 100. Thus, embodiments of the present invention provide for users to not only passively view breathing system metrics, but to also actively manage functions and features of the portable breathing system 10.

In addition, the portable breathing system 10 may be used by medical users to communicate directly, in real-time, with first-responder users via the communications interface of the portable breathing system. In such an embodiment, medical users could provide medical treatment instructions to the first-responder users. Similarly, the first-responder users could use the communications port to ask questions or provide updates to the medical users. For instance, a doctor monitoring the breathing system metrics associated with a patient user may observe, in real-time, that the patient user's heart rate and blood pressure are rising rapidly. The doctor may then communicate, in real-time, with the first-responder user, via the portable breathing system's communication interface, and instruct the first-responder user to administer the patient user with a drug that may control the patient user's heart rate and blood pressure. Such communications may be performed via the communications interface of the portable breathing system 10 over the communications network 56, such as through a cellular network.

Although the invention has been described with reference to the preferred embodiment(s), it is noted that equivalents may be employed and substitutions made herein without departing from the scope of the invention.

Having thus described various embodiments of the invention, what is claimed as new and desired to be protected by Letters Patent includes the following:

1. A portable breathing system for allowing a first-responder user to provide positive airway pressure to a patient undergoing a pulmonary medical emergency, comprising:
a housing;
a power source for providing electrical power to the portable breathing system, with the power source positioned within the housing;
a fluid pump for generating the positive airway pressure provided to the patient, with the fluid pump positioned within the housing;
a controller for controlling the fluid pump, with the controller housed within the housing,
wherein the fluid pump is operable in both a bi-level positive airway pressure mode and a continuous positive airway pressure mode;
one or more sensors for sensing breathing system metrics;
a memory element for storing the sensed breathing system metrics, with the memory element positioned within the housing; and
a communications interface configured for wirelessly transmitting the breathing system metrics to an external computing device located at a medical facility, with the communications interface positioned within the housing,
wherein the communications interface is further configured for wirelessly receiving communications from the external computing device located at the medical facility.

2. The portable breathing system of claim 1, wherein the breathing system metrics are comprised of metrics associated with the portable breathing system and metrics associated with the patient.

3. The portable breathing system of claim 2, with the system further including—
an air filter;
an oxygen source;
a humidity source; and
a medication source.

4. The portable breathing system of claim 3, wherein the metrics associated with the components of the breathing system include one or more of the following: a power source level, an air filter level, an oxygen source level, a humidity source level, and a medication source level.

5. The portable breathing system of claim 2, wherein the metrics associated with the patient include one or more of the following: a breathing rate, a breathing tidal volume, an oxygen saturation level, a carbon dioxide saturation level, a pulse, and a blood pressure.

6. The portable breathing system of claim 1, wherein the communications received from the medical user are instructions for providing medical care to the patient.

7. The portable breathing system of claim 1, wherein the communications interface is further configured for wirelessly receiving control information for providing instructions to the controller for controlling the fluid pump.

8. The portable breathing system of claim 1, wherein the control information is operable to instruct the fluid pup to operate in either the bi-level positive airway pressure mode or the continuous positive airway pressure mode.

9. A method for monitoring breathing system metrics obtained from a portable breathing system, comprising the following steps:
providing the portable breathing system, with the portable breathing system including—
a fluid pump,
a controller for controlling the fluid pump, and
a communications interface for wirelessly transmitting breathing system metrics;
receiving the breathing system metrics from the portable breathing system;
monitoring the breathing system metrics to determine whether a metric violates a predefined threshold; and
upon the metric violating the predefined threshold, sending a notification to one or more users.

10. The method of claim 9, wherein the breathing system metrics are comprised of metrics associated with the breathing system and associated with the patient.

11. The method of claim 10, wherein the metrics associated with the components of the breathing system include one or more of the following: a power source level, an air filter level, an oxygen source level, a humidity source level, and a medication source level.

12. The portable breathing system of claim 9, wherein the metrics associated with the patient include one or more of the following: a breathing rate, a breathing tidal volume, an oxygen saturation level, a carbon dioxide saturation level, a pulse, and a blood pressure.

13. The method of claim 9, further including the step of sending control information to the controller for controlling the fluid pump in response to the metric falling below the predefined threshold.

14. A portable breathing system for controlling an amount of oxygen provided to a patient, comprising:
a housing;
a power source for providing electrical power to the breathing system, with the power source positioned within the housing;
an oxygen source fluidly connected with the housing, with the oxygen source positioned outside of the housing;
a fluid pump for providing oxygen from the oxygen source and ambient air to the patient, with the fluid pump positioned within the housing;
a controller for controlling the fluid pump, with the controller housed within the housing,
an oxygen sensor for measuring an amount of oxygen provided to the patient via the fluid pump, with the oxygen sensor in communication with the controller; and
a pulse oximeter for monitoring an oxygen saturation level of the patient, with the pulse oximeter in communication with the controller;
wherein the controller is operable to control the amount of oxygen provided to the patient, via the fluid pump, based on the oxygen saturation level of the patient measured by the pulse oximeter.

15. The portable breathing system of claim 14, further including a user interface, such that a user can set the amount of oxygen provided to the patient.

16. The portable breathing system of claim 14, wherein the controller includes a feedback circuit for controlling the amount of oxygen provided to the patient based on the oxygen saturation level of the patient.

17. The portable breathing system of claim 16, wherein upon the oxygen saturation level of the patient falling below a predefined threshold, the controller instructs the fluid pump to increase the amount of oxygen supplied to the patient.

18. The portable breathing system of claim 16, wherein upon the oxygen saturation level of the patient rising above the predefined threshold, the controller instructs the fluid pump to decrease the amount of oxygen supplied to the patient.

19. The portable breathing system of claim 14 further including—
a memory element for storing breathing system metrics; and
a communications interface configured for wirelessly transmitting the breathing system metrics to an external computing device located at a medical facility.

20. The portable breathing system of claim 19, wherein the breathing system metrics includes one or more of the following: the amount of oxygen provided to the patient, the breathing rate of the patient, the breathing tidal volume of the patient, and an oxygen level of the patient.
